(19) United States
(12) Plant Patent Application Publication
(10) Pub. No.: US 2010/0138968 P1
(43) Pub. Date: Jun. 3, 2010

(54) Erysimum plant named 'Sapery'

(50) ;

(76) Inventor: Stephen Membrey, Frankston (AU)

Correspondence Address: MARK P. BOURGEOIS, 2851 Rockinghorse Lane, Niles, MI, 49120, US

(21) Appl. No.: 12/315,126

(22) Filed: Nov. 29, 2008

(51) Int. Cl. A01H 5/00

(52) U.S. Cl. PLT/263.1

(57) ABSTRACT

A new cultivar of Erysimum plant named ‘Sapery’ that is characterized by short height, a compact habit, green leaves with white hairs and violet flowers.

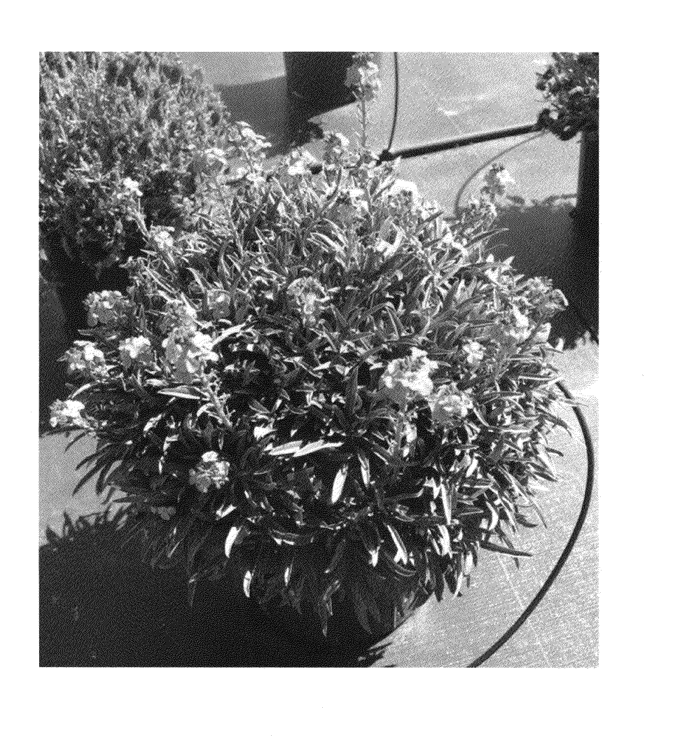

### BOTANICAL CLASSIFICATION

Erysimum linifolium

### VARIETY DENOMINATION

‘Sapery’

### BACKGROUND OF THE INVENTION

The present invention relates to a new and distinct cultivar of Erysimum plant botanically known as Erysimum linifolium and hereinafter referred to by the cultivar name ‘Sapery’.

‘Sapery’ was discovered as a naturally occurring whole plant mutation in a population of commercially grown Erysimum linifolium ‘Bowles Mauve’ (not patented) plants in a cultivated area of Dromana, Victoria, Australia in 2004. ‘Sapery’ was selected as a single plant.

Asexual reproduction of the new cultivar ‘Sapery’ first occurred by terminal cuttings in 2004 in Dromana, Victoria, Australia.

Since that time, under careful observation, the unique characteristics of the new cultivar have been uniform, stable and reproduced true to type in successive generations of asexual reproduction.

### SUMMARY OF THE INVENTION

The following represent the distinguishing characteristics of the new Erysimum cultivar ‘Sapery’. These traits in combination distinguish ‘Sapery’ as a new and distinct cultivar apart from other existing known varieties of Erysimum.
1. Erysimum ‘Sapery’ exhibits a short plant height and a compact habit.
2. Erysimum ‘Sapery’ exhibits green leaves with white hairs.
3. Erysimum ‘Sapery’ exhibits violet colored flowers.

The closest comparison cultivar is Erysimum ‘Bowles Mauve’.

‘Sapery’ is distinguishable from ‘Bowles Mauve’ by the following characteristics:
1. ‘Sapery’ grows to one-half the height of ‘Bowles Mauve’.
2. ‘Sapery’ has shorter peduncles than ‘Bowles Mauve’.

### BRIEF DESCRIPTION OF THE DRAWING

The accompanying photograph illustrates the distinguishing traits of Erysimum ‘Sapery’. The plant in the photograph shows an overall view of a 20 week old plant. The photograph was taken using conventional techniques and although colors may appear different from actual colors due to light reflectance it is as accurate as possible by conventional photographic techniques.

### BOTANICAL DESCRIPTION OF THE PLANT

The following is a detailed description of the new Erysimum cultivar named ‘Sapery’. Data was collected in Dromana, Victoria, Australia from 20 week old outdoor grown plants in 1 liter containers. The time of year was Winter in the Southern Hemisphere. Color determinations are in accordance with The Royal Horticultural Society Colour Chart 2007 edition, except where general color terms of ordinary dictionary significance are used. The growing requirements are similar to the species. ‘Sapery’ has not been tested under all possible conditions and phenotypic differences may be observed with variations in environmental, climatic, and cultural conditions, however, without any variance in genotype.
Botanical classification: Erysimum linifolium ‘Sapery’.
Use: Ornamental Perennial.
Parentage: A naturally occurring whole plant mutation of Erysimum linifolium ‘Bowles Mauve’.
Vigor: Moderate.
Growth habit: Upright.
Plant shape: Rounded.
Suitable container size: 1 liter.
Height: 30 cm. in height.
Width: 30 cm. in width.
Low temperature tolerance: 2° Centigrade.
High temperature tolerance: 30° Centigrade.
Propagation: Terminal cuttings.
Root system: Fibrous.
Stem:
   Branching habit.—Free branching.
   Average number of lateral branches.—36.
   Stem diameter.—4 mm. in diameter.
   Stem length.—20 cm. in length.
   Stem strength.—Strong.
   Stem color.—138B.
   Internode length.—4 mm. between nodes.
Foliage:
   Texture.—Smooth.
   Leaf arrangement.—Alternate.
   Compound or single.—Single.
   Leaf shape.—Obolanceolate.
   Leaf base.—Attenuate.
   Leaf tip.—Acute.
   Leaf margin.—Entire.
   Leaf color (lower surface).—137B.
   Leaf color (upper surface).—137B.
   Pubescence.—Covered with fine white hairs, color N155A.
   Leaf attachment.—Sessile.
Flower:
   Flowering habit.—Continuous from July to October in the Southern Hemisphere.
   Inflorescence type.—Raceme.
   Inflorescence length.—12 cm. in length.
   Inflorescence diameter: 5 cm. in diameter.
   Quantity of flowers per inflorescence.—12.
   Quantity of flowers and buds per plant.—90.
   Lastingness of flowers.—10 days.
   Bud shape.—Elliptic.
   Bud length.—8 mm. in length.
   Bud diameter.—4 mm. in diameter.
   Bud color.—85A.
   Fragrance.—None.
   Flower shape.—Rotate.
   Flower dimensions.—Average 1.5 cm. in diameter and 1.4 cm. in height.
   Petal texture.—Velvety, soft.
   Number of petals.—4.
   Fused or unfused.—Unfused.
   Petal shape.—Orbicular.
   Petal margin.—Entire.
   Petal apex.—Obtuse.
   Petal length.—1.2 cm.
   Petal width.—0.8 cm.
   Petal color (both surfaces).—85B.
   Self-cleaning or persistent.—Self-cleaning.
   Number of sepals.—4.
   Sepal shape.—Lanceolate.
   Sepal margin.—Entire.
   Sepal apex.—Acute.
   Sepal dimensions.—1 cm. in length and 2 mm. in width.
   Sepal color, (upper side).—137C.
   Sepal color, (under side).—137C.
   Peduncle dimensions.—30 cm. in length and 0.5 cm. in diameter.
   Peduncle strength.—Strong.
   Peduncle color.—138B.
   Pedicel dimensions.—5 mm. in length and 0.7 mm. in diameter.
   Pedicel color.—138B.
Reproduction organs:
   Stamen number.—5.
   Anther shape.—Ligulate.
   Anther dimensions.—2 mm. in length; filament 4 mm. in length.
   Anther color.—150B.
   Amount of pollen.—Low.
   Pollen color.—12B.
   Pistil number.—One in number.
   Pistil length.—7 mm.
   Stigma shape.—Rounded.
   Stigma color.—1B.
   Style length.—5 mm.
   Style color.—1B.
Fruit and seed production: Has not been observed.
Disease and pest resistance: Disease and pest resistance has not been observed.

1. A new and distinct variety of Erysimum plant named ‘Sapery’ as described and illustrated.
